(19) United States
(12) Patent Application Publication
(10) Pub. No.: US 2024/0390106 A1
(43) Pub. Date: Nov. 28, 2024

(54) DIAGNOSTIC MARKER TOOL FOR RADIOGRAPHIC OR MAGNETIC RESONANCE IMAGING

(71) Applicant: Vincent NESHAVA, MILLBROOK, SOUTHAMPTON (GB)

(72) Inventor: Vincent NESHAVA, MILLBROOK, SOUTHAMPTON (GB)

(21) Appl. No.: 18/688,007

(22) PCT Filed: Aug. 31, 2022
(86) PCT No.: PCT/GB2022/052220

§ 371 Date: Feb. 29, 2024

(30) Foreign Application Priority Data
Aug. 31, 2021 (GB) 2112396.3

(51) Int. Cl. A61B 90/00; A61B 90/94

(52) U.S. Cl. CPC A61B 90/39; A61B 90/94; A61B 2090/3966; A61B 2090/3991; A61B 2090/3995

(57) ABSTRACT

A diagnostic marker tool is provided for use with x-ray imaging and/or magnetic resonance imaging diagnostic methods. The diagnostic marker tool comprises a marker body having first and second opposed major surfaces; a first suction element positioned at the first major surface and a second suction element positioned at the second major surface. A non-metallic and/or radio-opaque indicator element is then associated with the marker body.

### RELATED APPLICATION

This application is based on prior filed copending International Application No. PCT/GB2022/052220 filed Aug. 31, 2022, which claims priority to Great Britain Application No. 2112396.3, filed Aug. 31, 2021, the entire subject matter of these applications is incorporated herein by reference in its entirety.

### TECHNICAL FIELD

The present invention relates to a diagnostic marker tool, particularly but not necessarily exclusively for use in both radiographic and magnetic resonance imaging contexts. The invention further relates to a kit comprising a pair of said diagnostic marker tools.

### BACKGROUND

In order to assist with the interpretation of x-ray images during medical processes, it is common in the art to provide markers associated with the x-ray imaging machinery which are radio-opaque, thereby leaving an imprint on the x-ray image produced. These markers can be used as indicators of the left and right sides of the patient to be imaged.

There are two types of markers which are used. The first comprises small metal pieces which have cut-out ‘L’ or ‘R’ indicia thereon. The metal impedes x-ray passage, and therefore appear as light patches on the x-ray image, with the ‘L’ or ‘R’ showing as dark characters. The metal pieces are typically hung on cloth strings or chains to allow for suspension of the markers on an x-ray receptor.

Alternatively, the metal pieces may sometimes be taped or tacked to the x-ray receptor.

The other marker type comprises a horseshoe-like metal bracket which can be hung over the x-ray receptor to produce the same effect.

With the former marker, the cloth or chain hangers are often worn by radiography staff, over their necks, and the cloth strings cannot be adequately sanitized between patient interactions, which can lead to disease transmission. The tape or tack which is used to hold the markers in place during a procedure is usually reused on multiple patients, thereby spreading infection. This can cause damage to the x-ray receptor over time, particularly if left in place for long durations.

With the second marker type, the horseshoe brackets are machine-specific; the dimensions of the marker must be bespoke to the depth of the x-ray receptor, and there is little prospect of cross-compatibility for different devices.

The markers are also higher unsuitable for fluoroscopic imaging, since the fluoroscope moves during the procedure which would cause either type of marker to fall off the x-ray receptor.

The use of metal components also prohibits the use of the markers in other radiographic contexts, particularly those relying on magnetic resonance.

### SUMMARY

The present invention seeks to provide a diagnostic marker tool for radiographic or resonance imaging which obviates or overcomes the above-mentioned deficiencies.

According to a first aspect of the invention, there is provided a diagnostic marker tool for use with x-ray imaging and/or magnetic resonance imaging diagnostic methods, the diagnostic marker tool comprising: a marker body having first and second opposed major surfaces; a first suction element positioned at the first major surface; a second suction element positioned at the second major surface; and a non-metallic and/or radio-opaque indicator element associated with the marker body.

The use of suction elements to mount diagnostic marker tools is advantageous over existing mounting means. The lack of adhesive significantly improves the ability to wipe, clean, and disinfect the diagnostic marker tool in use, whilst also allowing the diagnostic marker tools to be used on any diagnostic equipment with an appropriate flat mounting surface. The provision of double-sided suction ensures that the correct orientation of the diagnostic marker tool can be readily achieved, as sometimes, the facing of the tool must be reversed based on the imaging technique used and/or the patient orientation. Suction also provides.

Optionally, the marker body may include a shaped fluid chamber therein which receives a non-metallic and/or radio-opaque fluid to form the non-metallic and/or radio-opaque indicator element.

A shaped fluid chamber formed inside the marker body allows for the insertion of a suitable fluid to generate an indicator element. The fluid can be made bespoke to the context required, and usable in various alternate contexts.

Preferably, the shaped fluid chamber may include a removable cap to permit replacement of the non-metallic and/or radio-opaque fluid.

The ability to refill the shaped fluid chamber enables the user to alter the functionality of the diagnostic marker tool without needing to obtain a second tool for a different use. This advantageously reduces the equipment requirements for the user.

Optionally, an inlet of the shaped fluid chamber may be on an upper edge of the marker body.

Positioning the inlet on the upper edge of the marker body, with respect to a primary indicator thereof, ensures that the orientation of the diagnostic marker tool is easily identifiable, and that there is a reduced likelihood of leaking of the fluid from the shaped fluid chamber under gravity during use.

The non-metallic and/or radio-opaque fluid may comprise gadolinium for MRI-compatibility. The non-metallic and/or radio-opaque fluid may comprise barium sulphate or other radiographic contrast media for x-ray imaging compatibility.

Existing tools cannot be utilised with magnetic resonance imaging machinery, due to the metallic components. The present invention therefore provides ample opportunity for generalised used of the diagnostic marker tool with MRI machines as well as x-ray imaging machines.

Preferably, the marker body may be formed from a non-radio-opaque or radiolucent material.

Radio-opacity should be solely provided by the indicator element, and therefore it is preferred that the marker body itself does not interact with the imaging techniques utilised.

In a preferred embodiment, the marker body may be formed from a plastics or similarly radiolucent material. Plastics materials are easily sanitised, which allows for simple wipe-clean processing of the diagnostic marker tools between uses to avoid cross-contamination.

The diagnostic marker tool may further comprise an orientation indicator on the marker body for determining correct placement of the diagnostic marker tool in-use. One or more orientation markers can provide a visual indication to the user as to which way round the diagnostic marker tool should be, since the primary indicator may be obscured by the suction elements. This can not only be a top-bottom indicator, but could also be a left-right indicator. This reduces time wasted trying to determine the correct orientation of the tool prior to use.

Preferably, the marker body may include an aperture therethrough, the first and second suction elements being formed by a double-sided suction element received through the aperture.

The insertion of a double-sided suction element through an aperture of the marker body is the easiest means of providing double-sided suction capability, since the splayed suction cups of the double-sided suction element prevent easy dislodging thereof. Assembly and disassembly of the diagnostic marker tool is therefore straightforward, which simplifies the disinfection procedure.

The non-metallic and/or radio-opaque indicator element may preferably be a ‘left’ or ‘right’ indicator. In the majority of cases, the diagnostic marker tool will be used for the purposes of distinguishing between left and right sides of an image of a patient, thereby reducing the likelihood of errors during therapy at a later date. In one embodiment, the marker body may be disciform or cuboid.

A disciform or cuboid tool will sit comfortably in the user's hand, providing an ergonomic means of attaching the diagnostic marker tool to a surface without scratching their hands.

Preferably, the diagnostic marker tool may be devoid of a chain or hanger.

The chains and hangers used in the art will often cause artefacts to be generated in the x-ray images produced, and therefore elimination of these components is a great advantage for the present invention. Furthermore, chains and hangers, particularly ones formed from cloth, are excellent disease-transmission vectors, and therefore hygiene improvements are also achieved with the present invention.

Optionally, the first and second suction elements may have a width which is less than that of the marker body.

By not obscuring the very perimeter of the marker body, indicators, such as the directional indicators, can be visible to the user even if the primary indicator is obscured.

The first and second suction elements may be transparent or translucent.

If the user is able to see through the suction elements, then the need for secondary indicators may be obviated completely, as the user will be able to view the primary indicator.

Preferably, the marker body may be formed by an additive manufacturing process.

Three-dimensional printing is likely to be the simplest means by which the complex shaped fluid chamber can be formed internal to the marker body.

According to a second aspect of the invention, there is provided a diagnostic marker tool for use with x-ray imaging and/or magnetic resonance imaging diagnostic methods, the diagnostic marker tool comprising: a marker body; a mounting element for mounting the marker body to a surface of a diagnostic imaging device; and a shaped fluid-receiving chamber inside the marker body for receiving a non-metallic and/or radio-opaque indicator fluid therein to form a non-metallic and/or radio-opaque indicator element.

The provision of a shaped fluid receiving chamber inside of a marker body allows for filling and refilling of the diagnostic marker tool with an appropriate fluid, based on the task at hand. This greatly increases the utility of the tool for different contexts.

The diagnostic marker tool may further comprise a non-metallic and/or radio-opaque indicator fluid in the shaped fluid-receiving chamber.

According to a third aspect of the invention, there is provided a diagnostic marker kit comprising a pair of diagnostic marker tools in accordance with either the first or second aspects of the invention, wherein the non-metallic and/or radio-opaque indicator elements of the diagnostic marker tools are different to one another.

It will be apparent to the diagnostic technician that in many situations, it is best to provide paired markers on images to allow for certain definition of the respective left- and right-hand sides of an image. As such, paired tools as part of a kit of parts is much preferred.

### BRIEF DESCRIPTION OF THE DRAWINGS

The invention will now be more particularly described, by way of example only, with reference to the accompanying drawings, in which:

FIG. 1 shows a side perspective representation of a first embodiment of a diagnostic marker tool in accordance with the first aspect of the invention;

FIG. 2 shows a front perspective view of a second embodiment of a marker body of a diagnostic marker tool in accordance with the first aspect of the invention;

FIG. 3 shows a front view of the marker body of FIG. 2;

FIG. 4 shows a rear view of the marker body of FIG. 2;

FIG. 5 shows a cross-sectional view through the marker body of FIG. 2 showing the shaped fluid chamber;

FIG. 6 shows a front view of a third embodiment of a marker body of a diagnostic marker tool in accordance with the first aspect of the invention;

FIG. 7 shows a rear view of the marker body of FIG. 6; and

FIG. 8 shows a cross-sectional view through the marker body of FIG. 6 showing the shaped fluid chamber.

### DETAILED DESCRIPTION

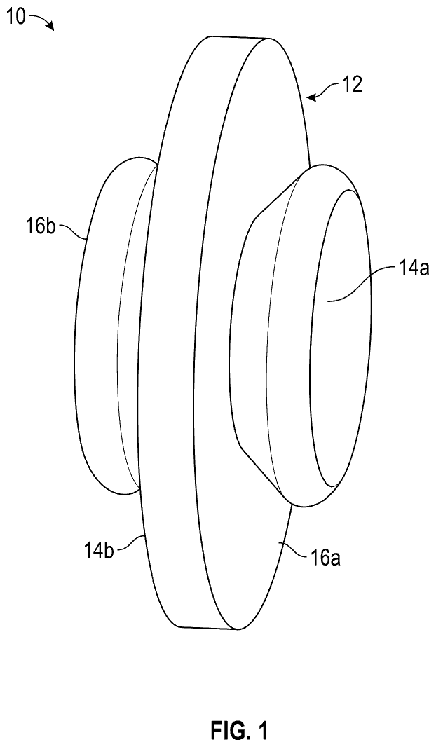

Referring to FIG. 1, there is indicated a diagnostic marker tool, referenced globally at 10, and which is suitable for use in radiographic and/or resonance imaging applications.

The diagnostic marker tool 10 comprises a marker body 12 which is preferably disciform in shape, though cuboidal tools could also be considered, having first and second major opposed surfaces 14a, 14b. These first and second major opposed surfaces 14a, 14b effectively form the front and rear sides of the diagnostic marker tool 10. The marker body 12 is preferably formed from a plastics material, but any appropriately non-radio-opaque material could be utilised.

The diagnostic marker tool 10 includes first and second suction elements 16a, 16b, thereby forming front and rear suction cups for the tool 10. The first and second suction cups 16a, 16b respectively extend from either of the first and second major opposed surfaces 14a, 14b.

The first and second suction elements 16a, 16b preferably have a maximum radial extent or width which is less than that of the marker body 12. This allows for visual inspection of a perimeter of the marker body 12 by a user during operation. In some cases, the use of clear transparent suction cups may overcome this limitation permitting the use of suction cups with a radial extent equal to or greater than that of the marker body.

The suction elements 16a, 16b permit mounting of the diagnostic marker tool 10 to many different surface types. If a flat or near flat surface is present on a piece of diagnostic equipment, then the diagnostic marker tool 10 can be mounted thereto.

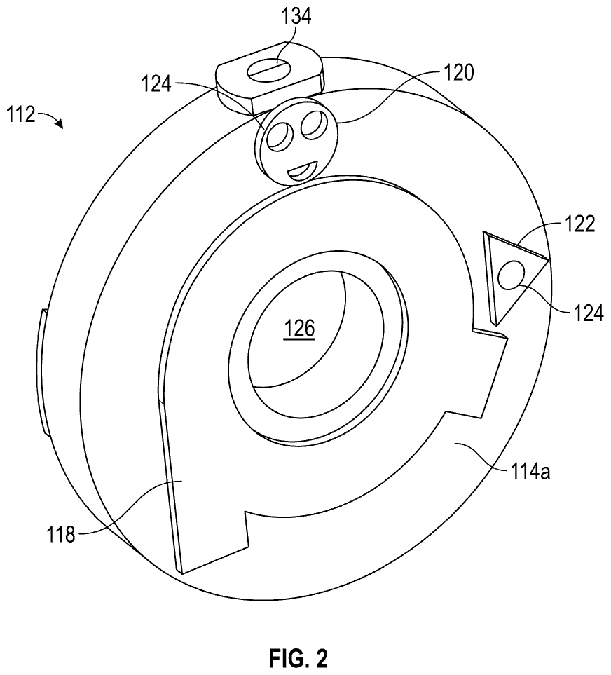

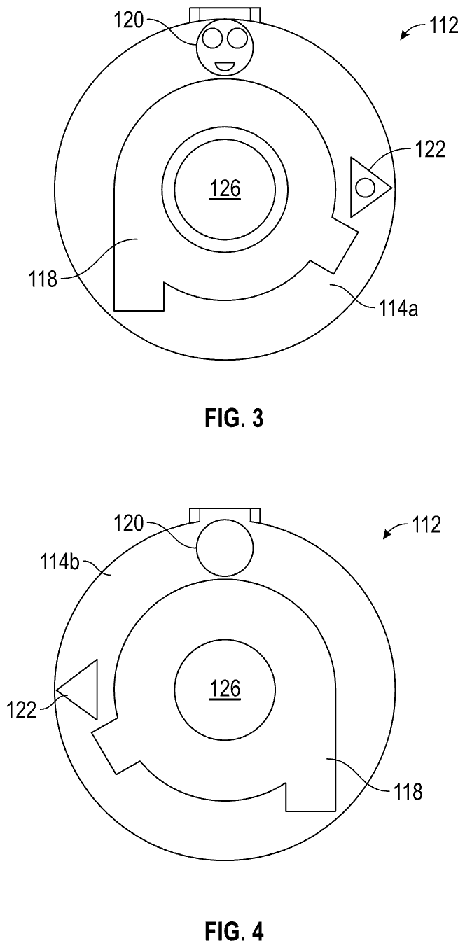

FIGS. 2 to 4 show an indicative marker body 112 of a diagnostic marker tool 110 for indicating a right-hand side of the image.

The marker body 112 includes a primary indicator 118 in the shape of a stylised ‘R’ on the first major surface 114a, with a mirrored primary indicator 118 on the second major surface 114b. The primary indicator 118 shows the position where an indicator will appear on any radiographic or magnetic resonance imaging image once used. It is extremely useful to provide a primary indicator 118 for the user's reference on the marker body 112, though the diagnostic marker tool will be functional without said.

Secondary indicia may also be provided, such as a top indicator 120, which is present on both of the first and second major surfaces 114a, 114b, or a side indicator 122, also present on both of the first and second major surfaces 114a, 114b. Further indicator portions 124 may be provided in order to show which of the first and second major surfaces 114a, 114b are the front and rear surfaces.

The marker body 112 includes a central aperture 126 with which the first and second suction elements engage. Preferably, the first and second suction elements are formed as an integral component, which can be forced through the central aperture 126 to produce the double-sided suction arrangement illustrated in FIG. 1.

When the suction elements are in place through the aperture, at least the orientation indicators, that is the top and side indicators 120, 122 should be visible. This is achieved by ensuring that the width or radius of the suction elements is sufficiently small compared with the width or radius of the marker body 112 to allow the top and side indicators 120, 122 to be positioned outwardly of the edge of the suction elements.

Furthermore, it is preferred that the suction elements be formed from a transparent or translucent material, so that the primary indicators 118 are visible therethrough. Of course, alternative types of primary indicator could be used; colour, for instance, could provide a means of differentiating between left and right tools very quickly. Additionally, or alternatively, primary indicators 118 could be put onto the circumference of the marker body 112, or around the perimeter thereof to be visible in the same manner as the top and side indicators 120, 122.

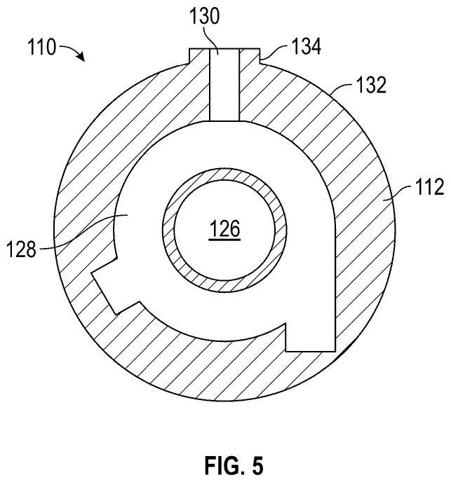

Nothing about the marker body 112 thus far described has been radio-opaque. FIG. 5 indicates how radio-opacity is achieved for the present embodiment. There is provided a shaped fluid chamber 128 inside the marker body 112 into which non-metallic and/or radio-opaque fluid can be introduced. This then forms a non-metallic and/or radio-opaque indicator element associated with the marker body 112.

The non-metallic and/or radio-opaque fluid may be replaceable, so that different fluids can be introduced. There is here illustrated an inlet 130 to the shaped fluid chamber 128, preferably extending to a circumference 132 of the marker body 112, via which the fluid can be introduced. The inlet 130 may be capped, for instance, with a screw-threaded cap 134 of the form visible in FIG. 2.

In the embodiment shown, the inlet 130 has a projecting portion 136 which extends beyond the circumference of the marker body 112. This may not only provide a simpler means of filling the shaped fluid chamber 128, but may also provide a directional indicator for the diagnostic marker tool 110. This inlet 130 is preferably provided on a top of the marker body 112, with the top side being made with reference to the orientation of the primary indicator 118.

The non-metallic and/or radio-opaque fluid may, by way of example only, comprise gadolinium for MRI-compatibility. Additionally or alternatively, the non-metallic and/or radio-opaque fluid may comprise barium sulphate or other radiographic contrast fluid for x-ray imaging compatibility.

The shaped fluid chamber 128 is in the form of a stylised ‘R’, thereby indicating that it relates to the right-hand diagnostic marker tool. The shaped fluid chamber 128 is matched in shape to the primary indicator 118, such that the primary indicator 118 identifies to a user the position of the non-metallic and/or radio-opaque indicator element.

A standard capital ‘R’ cannot be readily formed given the position of the central aperture 126, and therefore a stylised version is provided instead.

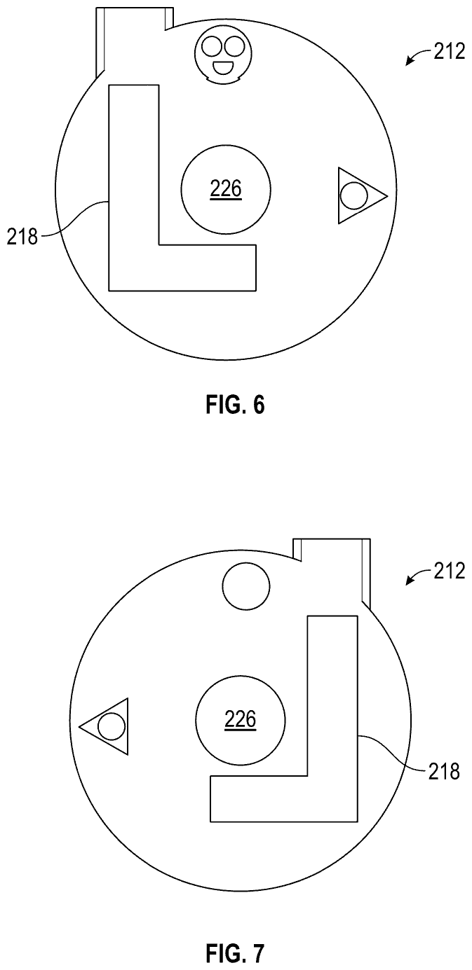

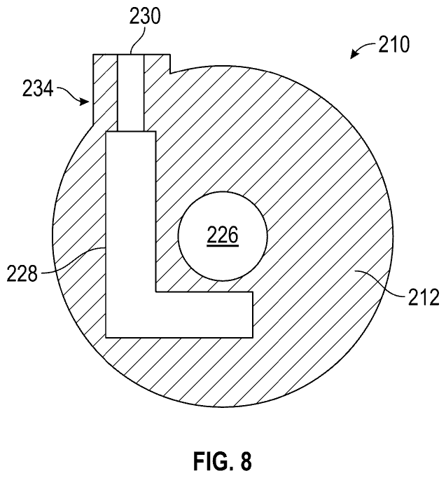

FIGS. 6 to 8 show the equivalent left-hand diagnostic marker tool 210, with the marker body being indicated at 212. Here, the inlet 230 is positioned to correspond with the top of the L-shaped shaped fluid chamber 228, and thus the corresponding projection 234 is offset centrally from the central aperture 226. Otherwise, the diagnostic marker tool 210 is equivalent to the right-hand version, albeit having an L-shaped primary indicator 218 and indeed, the two are intended to be used together kit comprising a pair of diagnostic marker tools, wherein the non-metallic and/or radio-opaque indicator elements of the diagnostic marker tools are different to one another.

To use the kit, the user affixes a suction element of one of the diagnostic marker tools to a left-hand side of the diagnostic imaging equipment being used, and a suction element of the other diagnostic marker tool to the right-hand side thereof. Patient orientation relative to the diagnostic imaging equipment may determine which way round the diagnostic marker tools are used. However, the paired suction elements of each diagnostic marker tool allow for the diagnostic marker tools to be affixed in any useful orientation.

The diagnostic marker tools of the present invention are described as having shaped fluid chambers for receiving non-metallic and/or radio-opaque indicator fluids therein. However, it will be appreciated that a suitable indicator element, such as an integrated solid opaque member, preferably an omnipaque material, could be built into the marker body of the diagnostic marker tool to achieve much the same effect. Such a diagnostic marker tool could still be mounted to different types of diagnostic equipment, including magnetic resonance imaging scanners, computerized tomography scanners, fluoroscopy scanners, and basic x-ray imagers.

It is noted that the diagnostic marker tools are intended to identify the left- and right-hand sides of the imaging apparatus, and thus the primary indicia are in the form of stylised ‘L’ and ‘R’ indicators. It will of course be apparent that any appropriate form of indicator could be used, either as an alternative means of identifying left- and right-hand sides, or to identify other relevant features. For example, up and down markers could be envisioned in some imaging arrangements.

Furthermore, it may be possible to put both the ‘L’ and ‘R’ indicators onto a single marker body, the respective non-metallic and/or radio-opaque indicator elements being formed together or separately on or in the marker body. This reduces the number of tools required to correctly distinguish between the left and right sides.

It is therefore possible to provide a diagnostic marker tool which is capable of being mounted to different types of imaging machine without interfering with the imaging capabilities. This can be achieved by use of suction elements on the marker body, thereby eliminating the metal components used in the art.

It is also possible to provide a diagnostic marker tool which has a shaped fluid-receiving chamber inside the marker body for receiving a non-metallic and/or radio-opaque indicator fluid therein to form a non-metallic and/or radio-opaque indicator element. This again assists with the elimination of metal components from the diagnostic marker tool, whilst also allowing refilling of the tool with different fluids to alter its properties.

The words ‘comprises/comprising’ and the words ‘having/including’ when used herein with reference to the present invention are used to specify the presence of stated features, integers, steps, or components, but do not preclude the presence or addition of one or more other features, integers, steps, components, or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

The embodiments described above are provided by way of examples only, and various other modifications will be apparent to persons skilled in the field without departing from the scope of the invention as defined herein.

1. A diagnostic marker tool for use with x-ray imaging and/or magnetic resonance imaging diagnostic methods, the diagnostic marker tool comprising:
a marker body having first and second opposed major surfaces;
a first suction element positioned at the first major surface;
a second suction element positioned at the second major surface; and
a non-metallic and/or radio-opaque indicator element associated with the marker body.

2. A diagnostic marker tool as claimed in claim 1, wherein the marker body includes a shaped fluid chamber therein which receives a non-metallic and/or radio-opaque fluid to form the non-metallic and/or radio-opaque indicator element.

3. A diagnostic marker tool as claimed claim 2, wherein the shaped fluid chamber includes a removable cap to permit replacement of the non-metallic and/or radio-opaque fluid.

4. A diagnostic marker tool as claimed claim 2, wherein an inlet of the shaped fluid chamber is on an upper edge of the marker body.

5. A diagnostic marker tool as claimed claim 2, wherein the non-metallic and/or radio-opaque fluid comprises gadolinium for MRI-compatibility.

6. A diagnostic marker tool as claimed in claim 2, wherein the non-metallic and/or radio-opaque fluid comprises barium sulphate or other radiographic contrast media for x-ray imaging compatibility.

7. A diagnostic marker tool as claimed in claim 1, wherein the marker body is formed from a radiolucent material.

8. A diagnostic marker tool as claimed in claim 7, wherein the marker body is formed from a plastics material.

9. A diagnostic marker tool as claimed in claim 1, further comprising an orientation indicator on the marker body for determining correct placement of the diagnostic marker tool in-use.

10. A diagnostic marker tool as claimed in claim 1, wherein the marker body includes an aperture therethrough, the first and second suction elements being formed by a double-sided suction element received through the aperture.

11. A diagnostic marker tool as claimed in claim 1, wherein the non-metallic and/or radio-opaque indicator element is a ‘left’ or ‘right’ indicator.

12. A diagnostic marker tool as claimed in claim 1, wherein the marker body is disciform.

13. A diagnostic marker tool as claimed in claim 1, wherein the diagnostic marker tool is devoid of a chain or hanger.

14. A diagnostic marker tool as claimed in claim 1, wherein the first and second suction elements have a width which is less than that of the marker body.

15. A diagnostic marker tool as claimed in claim 1, wherein the first and second suction elements are transparent or translucent.

16. A diagnostic marker tool as claimed in claim 1, wherein the marker body is formed by an additive manufacturing process.

17. A diagnostic marker tool for use with x-ray imaging and/or magnetic resonance imaging diagnostic methods, the diagnostic marker tool comprising:
a marker body;
a mounting element for mounting the marker body to a surface of a diagnostic imaging device; and
a shaped fluid-receiving chamber inside the marker body for receiving a non-metallic and/or radio-opaque indicator fluid therein to form a non-metallic and/or radio-opaque indicator element.

18. A diagnostic marker tool as claimed in claim 17, further comprising a non-metallic and/or radio-opaque indicator fluid in the shaped fluid-receiving chamber.

19. A diagnostic marker kit comprising a pair of diagnostic marker tools as claimed in claim 1, wherein the non-metallic and/or radio-opaque indicator elements of the diagnostic marker tools are different to one another.
